(19) United States
(12) Patent Application Publication
(10) Pub. No.: US 2023/0165989 A1
(43) Pub. Date: Jun. 1, 2023

(54) AN ACTIVATABLE WOUND DRESSING

(71) Applicant: AGA Nanotech Ltd, Watford Hertfordshire (GB)

(72) Inventor: Adrian Fellows, Watford Hertfordshire (GB)

(73) Assignee: AGA Nanotech Ltd, Watford Hertfordshire (GB)

(21) Appl. No.: 17/904,965

(22) PCT Filed: Feb. 23, 2021
(86) PCT No.: PCT/EP2021/054400

§ 371 Date: Aug. 25, 2022

(30) Foreign Application Priority Data
Feb. 28, 2020 (GB) 2002836.1
Jul. 7, 2020 (GB) 2010402.2

(51) Int. Cl. A61L 2/18; A61F 13/00

(52) U.S. Cl. CPC A61L 2/186; A61F 13/00068

(57) ABSTRACT

The invention relates to a wound dressing or other wound contact medium containing an acetyl donor compound, and being free from peroxide compounds which is activatable by a means of applying hydrogen peroxide. The wound dressing or other wound contact medium is suitable for use in the prevention and/or treatment of wound infection in a patient comprising applying the wound dressing or other wound contact medium to a wound of the patient, and subjecting the wound dressing or other wound contact medium to a means of applying hydrogen peroxide so that the acetyl donor compound of the wound dressing or other wound contact medium is activated by the hydrogen peroxide to produce peracetic acid.

The present invention relates to a wound dressing or other wound contact medium suitable for use in the treatment of wound infections of humans or animals, and a kit comprising the wound dressing or other wound contact medium, and a means of applying hydrogen peroxide, and a process for preventing and/or treating a wound infection in a patient.

Antibiotic resistance, particularly the emergence of widespread multiple drug resistant infections, poses a catastrophic risk to human health and involves substantial costs. Novel approaches to combat infection are therefore urgently required.

WO 2015/150722 relates to a therapeutic agent comprising micro- and/or nano-particle loaded with at least one inert precursor chemical for use in the treatment of an infection of a human or animal. The precursor chemical or chemicals are activatable by the physiological milieu in situ at the site of the infection to form an antimicrobial agent. In one embodiment, a peroxygen donor and an acetyl donor are used in combination as precursor chemicals. Upon activation, the peroxygen donor can form hydrogen peroxide which vice versa can activate the acetyl donor to form peracetic acid in situ.

Hydrogen peroxide and peracetic acid have a powerful biocidal effect on microorganisms. Thus, the therapeutic agent of WO 2015/150722 allows for generation of antimicrobial agents in situ at the site of infection so that an effective treatment of an infection can be provided. The storage stability of these therapeutic agents, however, leaves room for improvement. This is because the precursor chemicals, in particular the peroxygen donors, are reactive compounds so that spontaneous reaction of the chemicals may already occur during storage which could affect the properties of the agent and shorten storage life, or require more rigid storage conditions, e.g. (stronger) cooling.

It is an object of the present invention to provide a therapeutic system suitable for use in the prevention and/or treatment of wound infections of humans or animals, including multiple drug resistant infections, which at the same time has an excellent shelf life.

According to the present invention there is provided a wound dressing or other wound contact medium containing an acetyl donor compound, and being free from peroxide compounds. The invention also provides the wound dressing or other wound contact medium according to the invention for use in the prevention and/or treatment of wound infection in a patient comprising applying the wound dressing or other wound contact medium to a wound of the patient, and subjecting the wound dressing or other wound contact medium to a means of applying hydrogen peroxide. Thereby the acetyl donor compound of the wound dressing or other wound contact medium can be activated by the hydrogen peroxide to produce peracetic acid in situ.

Since the wound dressing or other wound contact medium does not contain reactive peroxide compounds, the storage stability is excellent and significantly improved compared to systems in which peroxide compounds are included. At the same time, generation of peracetic acid is possible in situ by activation with a means of applying hydrogen peroxide. Thus, peracetic acid known to have a powerful biocidal effect can be provided in situ at the site of the wound so that an effective prevention and/or treatment of a wound infection is possible. The inventive wound dressing or other wound contact medium delivers benefits in terms of potency of antimicrobial activity at site of need; enhanced activity against wound biofilm, and control of reactive oxygen and nitrogen species within the wound, in toto producing better wound healing.

The acetyl donor compound, optionally after release from particles, can react with hydrogen peroxide produced by the plasma as discussed below to produce peracetic acid or a mixture of peracetic acid and hydrogen peroxide, respectively. The use of an inert acetyl donor compound that can be activated by a means of applying hydrogen peroxide in situ overcomes problems of stability and safety for the active antimicrobial agent. In the present invention, a combination of wound dressing or other wound contact medium containing an acetyl donor and a means of applying hydrogen peroxide is used to produce peracetic acid or dynamic equilibrium mixtures of hydrogen peroxide and peracetic acid in situ on the wound site. Particularly, peracetic acid is known to be highly effective in disrupting biofilms and killing otherwise resistant organisms therein. Wound biofilms have been shown to be of consequence in preventing wound healing, especially in chronic wounds.

The system is also flexible in that it is possible to adjust the means of applying hydrogen peroxide such as concentration, and duration for each individual application. The peracetic acid concentration generated at the wound site can be adjusted irrespective from the wound dressing used.

The type of wound dressing can be selected from common wound dressings. Suitable natural materials include: gelatin; agarose; hypromellose; Matrigel; extracellular matrix proteins such as fibrin, fibronectin, collagen and collagen derivatives; polysaccharides, such as xanthan gum; sugars; celluloses and modified celluloses such as hydroxypropylcellulose, sodium carboxymethyl cellulose and hydroxyethyl cellulose; and polycarboxylic acids.

Other preferred wound dressings are on-porous and/or porous and cross-linked polymer and/or non-cross linked polymer material such as polyethylene oxide, polyvinyl alcohol, polyacrylic acid, polyvinyl pyrrolidone, polyacrylamidomethylpropanesulfonate, polycaprolactone (PCL), polyglycolic acid (and its derivatives) and copolymers thereof.

In some embodiments, the material comprises a commercial hydrogel selected from the group consisting of: AquaformIM, CurafilIM, Granugel™, Hypergel™, Intrasite GelIM, Nu-Gel™, and Purolin gelIM (Jones and Vaughan, 2005).

In other embodiments, the material comprises a polymeric material selected from the group consisting of: poly(lactide-co-glycolide), poly(vinyl pyrrolidone), polyvinyl alcohol), poly(hydroxyalkylmethacrylates), polyurethane-foam, and hydrocolloid and aliginate dressings (Boateng et al., 2008).

Other commercially available amorphous hydrogels that can be used include: Anasept™ Antimicrobial Skin & Wound Gel (Anacapa Technologies, Inc.), 3M™ Tegaderm™ Hydrogel Wound Filler (3M Health Care), AmeriDerm Wound Gel (AmeriDerm Laboratories, Ltd.), AquaSite™ Amorphous Hydrogel Dressing (Derma Sciences, Inc.), Curasol™ Gel Wound Dressing (Smith & Nephew, Advanced Wound Biotherapeutics), Dermagran™ Amorphous Hydrogel Dressing (Derma Sciences, Inc.), DermaPlex™ Gel (MPM Medical, Inc.), DermaSyn™ (DermaRite Industries, LLC), DuoDERM™ Hydroactive Sterile Gel (ConvaTec), Excel™ Gel (MPM Medical, Inc.), Gentell Hydrogel (Gentell Wound and Skin Care), Hydrogel Amorphous Wound Dressing (McKesson Medical-Surgical), Hypergel™ Hypertonic Gel (Molnlycke Health Care US, LLC), INTRASITE* Gel Hydrogel Wound Dressing (Smith & Nephew, Inc.), Kendall™ Amorphous Hydrogel (Covidien), LipoGel™ (Progressive Wound Care Technologies, Inc.), MacroPro™ Gel (Molnlycke Health Care US, LLC), MPM Regenecare™ HA Spray (MPM Medical, Inc.), Normlgel™ Isotonic Saline Gel (Molnlycke Health Care US, LLC), Purilon™ Gel (Coloplast Corp.), Regenecare™ HA (MPM Medical, Inc.), Restore™ Hydrogel (Amorphous) (Hollister Wound Care), SAF-Gel™ Hydrating Dermal Wound Dressing (ConvaTec), SilvaSorb™ Gel (Medline Industries, Inc.), SilverMed™ Amorphous Hydrogel (MPM Medical, Inc.), SilvrSTAT™ Antibacterial Wound Dressing Gel (ABL Medical, LLC), Skintegrity™ Hydrogel (Medline Industries, Inc.), SOLOSITE™ Wound Gel (Smith & Nephew, Inc.), Spand-Gel™ Primary Hydrogel (Medi-Tech International Corp.), and Woun'Dres™ Collagen Hydrogel (Coloplast Corp.).

In some embodiments, the hydrogel is in the form of a coating on a gauze pad, nonwoven sponge, rope and/or strip. In these embodiments, the screen comprises an impregnated hydrogel in which the hydrogel is coated onto a gauze pad, nonwoven sponge, rope and/or strip. The impregnated hydrogel may be formed by coating a gauze, sponge, rope or strip material with a suitable hydrogel, such as gelatin.

Alternatively, a commercially available impregnated hydrogel of this type that can be used, such as: AquaSite™ Hydrogel Impregnated Gauze (Derma Sciences, Inc.), DermaGauze™ (DermaRite Industries, LLC), Gentell Hydrogel Impregnated Gauze (Gentell Wound and Skin Care), Hydrogel Impregnated Gauze Dressing (McKesson Medical-Surgical), Kendall™ Hydrogel Impregnated Gauze (Covidien), MPM GelPad™ Hydrogel Saturated Gauze Dressing (MPM Medical, Inc.), Restore™ Hydrogel Dressing (Impregnated Gauze) (Hollister Wound Care), Skintegrity™ Hydrogel Dressing (Medline Industries, Inc.), and SOLOSITE™ Conformable Wound Gel Dressing (Smith & Nephew, Inc.).

In some embodiments, the dressing comprises a sheet hydrogel in which a hydrogel is supported by a thin fibre mesh. The sheet hydrogel may be formed by coating a fibre mesh with a suitable hydrogel, such as gelatin, Alternatively, a commercially available sheet hydrogel can be used, such as: AquaClear® (Hartmann USA, Inc.), AquaDerm™ (DermaRite Industries, LLC), Aquaflo™ Hydrogel Dressing (Covidien), AquaSite™ Hydrogel Sheet (Derma Sciences, Inc.), Aquasorb™ and Border (DeRoyal), Avogel™ Hydrogel Sheeting for Scars (Avocet Polymer Technologies, Inc.), Comfort-Aid™ (Southwest Technologies, Inc.), CoolMagic™ Gel Sheet (MPM Medical, Inc.), Curasol™ Gel Saturated 4×4 Dressing (Smith & Nephew, Advanced Wound Biotherapeutics), Derma-Gel™ Hydrogel Sheet (Medline Industries, Inc.), Elasto-Gel™ (Southwest Technologies, Inc.), FLEXIGEL* Hydrogel Sheet Dressing (Smith & Nephew, Inc.), Hydrogel Sheet Dressing (McKesson Medical-Surgical), MediPlus™ Barrier Gel Comfort Border (MediPurpose, Inc.), MediPlus™ Barrier Gel Hydrogel Dressing (MediPurpose®, Inc.) NU-GEL™ Wound Dressing (Systagenix), Spand-Gel™ Hydrogel Dressing Sheets (Medi-Tech International Corp.), Toe-Aid™ (Southwest Technologies, Inc.), and XCell™ Cellulose Wound Dressing (Medline Industries, Inc.).

In specific embodiments, the hydrogel is gelatin. Gelatin can be obtained by the hydrolysis of collagen by boiling skin, ligaments, tendons, etc. A mixture of 2% gelatin in water forms a stiff hydrogel. The hydrogel may be formed by adding gelatin to water at an elevated temperature to dissolve the gelatin. The solution is then cooled and the solid gelatin components form submicroscopic crystalline particle groups which retain a considerable amount of water in the interstices.

The hydrogel will typically be transparent but it may also be opalescent.

In still other embodiments, the wound dressing may comprise a biological dressing (e.g. hyaluronic acid, chitosan and elastin) or a synthetic polymer (e.g. gauze or polysiloxanes) or a combination of both (e.g. Integra1 M bilayer matrix wound dressing).

In still other embodiments, the wound dressing may comprise GanuGEL® ConVaTec.

Examples of other wound contact media of particular relevance in deep or awkwardly shaped wounds are plastic, textile or foam plugs or other conformable structures as well as gels, creams, foams or caulks.

The wound dressing or the other wound contact medium is free from peroxide compounds, i.e. it does not contain any peroxide compound. As known by the skilled person, peroxide compounds are compounds, which include a peroxo group (—O—O—) or the peroxide anion (O₂²⁻). Typical examples for peroxide compounds are hydrogen peroxide, peroxy acids such as percarbonates, perphosphates, perborates, or persulfates, metal peroxides such as sodium peroxides and lithium peroxide, and organic peroxides such as urea peroxide, peresters, and di-tert.-butyl peroxide.

In the context of the present invention, the term “free from peroxide compounds” is to be understood that there is no deliberate addition of peroxide compounds to the wound dressing according to the present invention. In preferred embodiment, the term “free from peroxide compounds” is to be understood that the wound dressing according to the present invention does not contain peroxide compounds in a significant amount.

It goes without saying that the feature “free from” or “free from peroxide compounds” only refers to the wound dressing or the other wound contact medium as produced or marketed and before use thereof. As will be discussed below, during use the wound dressing or the other wound contact medium is subjected to a means of applying hydrogen peroxide before and/or after applying it on the wound resulting in the in situ generation of peracetic acid.

It is preferred that the wound dressing or the other wound contact medium is also free from other compounds which can liberate oxygen upon activation and/or free from other peroxygen donors which form hydrogen peroxide upon activation. Accordingly, it is preferred that the wound dressing or the other wound contact medium is also free from superoxide compounds, dioxygenyl compounds, and ozone compounds.

The wound dressing or the other wound contact medium contains an acetyl donor compound. Preferably, the acetyl donor compound is substantially insoluble and inert. This insolubility and inertness provides the wound dressing or other wound contact medium according to the present invention with an even higher stability and further improved shelf-life. An acetyl donor compound can react with hydrogen peroxide to form peracetic acid, preferably on the addition of water. The acetyl donor preferably comprises any or a combination of the compounds in the following list:

### Tetraacetylethylenediamine (TAED)

Methyl cellulose encapsulated TAED or encapsulated donors
Acetyl salicylic acid (ASA)
Diacetyl dioxohexahydratriazine (DADHT)
Tetraacetyl glycoluril
Acetyl urea
Di-acetyl urea
Tri-acetyl urea
Pentaacetyl glucose (PAG)
Tetraacetyl glycoluril (TAGU)
Acetyl phosphate
Acetyl imidazole

### Acetyl CoA

Acetic anhydride
Compounds containing a hemiacetal group
Acetic acid
Di-, acetylmorphine

### Pyruvate

Acetyl chloride

### Acetyl-caprolactam

N′N′-Diacetyl-N′N′-dimethyl urea.

In a preferred embodiment, the acetyl donor compound is selected from tetraacetylethylenediamine (TAED), pentaacetyl glucose (PAG), acetyl salicylic acid (ASA), or a mixture of these. PAG includes alpha-PAG and beta-PAG. The use of alpha-PAG, beta-PAG, TAED and combinations of TAED and PAG (TAED in combination with alpha-PAG and TAED in combination with beta-PAG) as the acetyl donor is especially preferred.

In a preferred embodiment, the acetyl donor compound is contained in particles so that the wound dressing or other wound contact medium contains particles in which the acetyl donor compound is included. It is particular preferred that the acetyl donor compound is encapsulated within polymeric particles. The polymer for the polymer particles is preferably poly(lactic-co-glycolic acid) (PLGA). The particles, in particular the polymeric particles, are preferably microparticles and/or nanoparticles. Loading of the particles as indicated above with the acetyl donor compound can be achieved by known techniques either during the particle fabrication process or afterwards.

In case of acetyl donor compounds contained in particles, a release of the acetyl donor compounds occurs when the particle bursts, degrades or changes its porosity in situ, which may be on the body of the human or animal host, in particular in the body fluids in the wound to be treated. Advantageously, the particles degrades via hydrolysis over time to provide a controlled release of the acetyl donor compound.

Examples of suitable particles, in particular micro- and/or nano-particles, for use in the present invention are micelles, dendrimers, buckyballs, liposomes, ethosomes, mesoporous silica and nano-carbon tubes, all of which are capable of encapsulating other chemicals such as acetyl donor compounds.

Advantageously but not necessarily, the particles, preferably polymeric particles, in which the acetyl donor compound is encapsulated, especially the micro- and/or nano-particles, are produced by a thermally induced phase separation (TIPS) process. Such a process minimizes residues of solvents used in the encapsulation process that may otherwise compromise the safety and efficacy of the resulting particle. In addition, in some cases it is preferable for the particle to be biodegradable to produce harmless by-products. Preferably, therefore, the particle is comprised of a biodegradable polymer such as poly(lactic-co-glycolic acid) (PLGA) that can be used to produce particles, in particular micro- and/or nano-particles, encapsulating the acetyl donor compound by a TIPS process.

The release kinetics (rate and duration) can be modified by adjusting the composition of the polymer used to manufacture the polymer particles, such as micro- and nano-particles. The particles may be made of a variety of synthetic and natural polymers. Examples of such polymers are PLGA, poly(allylamine)hydrochloride, poly(diallylmethylammonium chloride), polyethylenimine (PEI), polyvinyl pyrollidone, poly L ornithine, poly L arginine, protamines, chitosan, alginates, polystyrene sulphonate, poly(acrylic acid), poly(methacrylic acid), polyvinylsulfonate, poly phosphoric acid, poly L glutamic acid, and dextran sulphate. Nanomicellular particles may also be made, for example, from polyethylene oxide/polypropylene oxide diblock and triblock copolymers, phospholipid or other surface active agents.

PLGA is the preferred polymer for the particles. PLGA is a copolymer that is synthesized by means of ring-opening co-polymerization of two different monomers, the cyclic dimers (1,4-dioxane-2,5-diones) of glycolic acid and lactic acid. It undergoes hydrolysis in vivo to produce the original monomers, lactic acid and glycolic acid, which under normal physiological conditions are by-products of various metabolic pathways in the body. Hence, there is minimal systemic toxicity associated with using PLGA for the purpose of the present invention.

As mentioned, polymeric particles in which the acetyl donor compound is encapsulated are preferably produced by a thermally induced phase separation (TIPS) process. However, persons skilled in the art will be aware that other methods of manufacture are possible.

The TIPS process begins with production of a polymer solution, e.g. a PLGA solution, at a high temperature in order to generate a homogenous solution. The acetyl donor compound is dissolved in a suitable solvent and is then blended into the polymer solution. The removal of thermal energy by rapid cooling below a biomodal solubility curve using another immiscible cooling liquid induces the phase de-mixing of the homogenous polymer solution into a multi-phase system containing a polymer-rich phase and polymer-lean phase. The phase separated polymer solution is subsequently treated by freeze-drying to remove the solvents, generating the particles, in particular micro- and/or nano-particles, suitable for the invention. A conventional microencapsulator can be used for the process, for example an Encapsulator VAR-D unit as manufactured by Nisco Engineering AG.

As indicated above, the particles in which the acetyl donor compound is contained, may comprise micro-particles, nano-particles or a mixture of the two. In accordance with the IUPAC (International Union of Pure and Applied Chemistry) definitions, micro-particles are particles of any shape with dimensions in the range of 1×10⁻⁷ m to 1×10⁻⁴ m whereas nano-particles are particles of any shape with dimensions in the range of 1×10⁻⁹ m to less than 1×10⁻⁷ m. Particle size and size distribution of micro- and nano-particle systems determine for instance the in vivo distribution, biological fate, toxicity and the targeting ability. In addition, they can also influence the drug loading, drug release and stability of the particle. The particle size is preferably less than 250 μm, e.g. 1 to less than 250 μm, since smaller particle sizes show a stronger oxidizing effect.

Poly(lactic-co-glycolic acid) (PLGA)—based particles can be produced over a size range from around 20 nm diameter up to micron sizes. The production of such particles is known and described, for example, in WO 2008/155558. The method of manufacture of these particles can be used to manipulate their properties such as size and surface to volume ratio, porosity, payload efficiency and drug release profile. This makes them particularly suited to being the particles used in this invention. Loading of these particles with the acetyl donor compound can be achieved by known techniques either during the particle fabrication process or afterwards.

The particles, such as polymeric particles, in which the acetyl donor compound is encapsulated, may be wetted, for instance by a surface active agent, e.g. pluronic acid. This is advantageous to work with the particles when they are hydrophobic and could be easily activated by the plasma within the hydrogel dressing.

The acetyl donor compound or the particles, in which the acetyl donor compound is contained, can be fixed in the wound dressing or other wound contact medium by any common measure. It is evident that the suitable measures strongly depend on the type of acetyl donor compound or particles used and the type of wound dressing or other wound contact medium used. In a preferred embodiment the wound dressing comprises a dressing matrix to which the particles are attached, e.g. by physical or chemical means. The particles are preferably attached to the dressing matrix via a linking group. In this regard, a bifunctional chemical compound can be used which can react with both the dressing matrix and the particles via the functional groups to provide a linking group therebetween.

The invention is also related to a wound dressing or other wound contact medium containing an acetyl donor for use in the prevention and/or treatment of wound infection in a patient.

The invention is also related to a wound dressing or other wound contact medium containing an acetyl donor for use in the prevention and/or treatment of wound infection in a patient comprising applying the wound dressing or other wound contact medium to a wound of the patient, and subjecting the wound dressing or other wound contact medium to a means of applying hydrogen peroxide so that the acetyl donor compound of the wound dressing or other wound contact medium is activated by the hydrogen peroxide to produce peracetic acid in situ.

The invention is also related to a wound dressing or other wound contact medium containing an acetyl donor and being free from peroxide compounds for use in the prevention and/or treatment of wound infection in a patient.

The invention is also related to a wound dressing or other wound contact medium containing an acetyl donor and being free from peroxide compounds for use in the prevention and/or treatment of wound infection in a patient comprising applying the wound dressing or other wound contact medium to a wound of the patient, and subjecting the wound dressing or other wound contact medium to a means of applying hydrogen peroxide so that the acetyl donor compound of the wound dressing or other wound contact medium is activated by the hydrogen peroxide to produce peracetic acid in situ.

The wound dressing or other wound contact medium of the invention is suitable for use in the prevention and/or treatment of wound infection in a patient. The wound dressing or other wound contact medium is as described above. The use comprises applying the wound dressing or other wound contact medium to a wound of the patient. A wound may be defined as a disruption in the continuity of the epithelial lining of the skin or mucosa and in the case of chronic open wounds such as diabetic foot ulcers or venous leg ulcers may be deep seated and involve tissues below the epidermis.

The use further comprises subjecting the wound dressing or other wound contact medium to a means of applying hydrogen peroxide. As a result, the acetyl donor compound of the wound dressing or other wound contact medium is activated by the hydrogen peroxide to produce peracetic acid in situ. Usually a mixture of hydrogen peroxide and peracetic acid is produced. The peracetic acid and optionally the hydrogen peroxide can exert their biocidal effect at the wound site.

In one embodiment, the means of applying hydrogen peroxide to the wound dressing or other wound contact medium is selected from
a means of applying hydrogen peroxide by spraying or soaking,
a means of applying hydrogen peroxide by padding with a hydrogen peroxide containing wipe or sterile pad,
a means of applying hydrogen peroxide by drip application,
a means of applying hydrogen peroxide by striping by passing over a series of holes in an applicator bar delivering the solution,
a means of applying hydrogen peroxide by printing a solution of gel containing hydrogen peroxide by gravure or other techniques,
a means of applying hydrogen peroxide in form of a vapour or mist.

Another means of applying hydrogen peroxide to the wound dressing or wound contact medium is by applying microorganisms which generate hydrogen peroxide in situ.

The concentration of the hydrogen peroxide applied to the wound dressing by the means of a hydrogen peroxide and can vary within a wide range. Hydrogen peroxide is readily available over the counter as a 3% or 6% solution and industrially at concentrations up to 35%. Supply of high concentrations has become restricted more recently because of potential use in explosive devices. The concentrations mentioned above are widely used in topical treatments and dental applications. They would generally be considered too high for application to open wounds. The appropriate dilution for use is likely to be much lower, in the millimolar range, and will be determined by a number of factors including the quantity of acetyl donor in the dressing, the nature of the wound, the duration of contact, the absorbency of the dressing and other factors. In one embodiment, the concentration of the hydrogen peroxide applied is whitin the range of 0.01 to 6 weight %, such as 0.01 to 1 weight % such as 0.01 to 3 weight %.

By subjecting the wound dressing or other wound contact medium with the means of applying hydrogen peroxide, an interaction between the dressing or other media and the plasma occurs. In particular, the acetyl donor compound of the dressing or other media is activated by or reacts with the hydrogen peroxide produced by the plasma to form peracetic acid in situ to impart biocidal properties at the wound site.

In one embodiment, the wound dressing or other wound contact medium is subjected to the means of applying hydrogen peroxide before it is applied to the wound. In one embodiment, the wound dressing or other wound contact medium is subjected to the means of applying hydrogen peroxide after it is applied to the wound. It is also possible to carry out said treatment with the means of applying hydrogen peroxide before and after it is applied to the wound. If the wound dressing or other wound contact medium is subjected to the means of applying hydrogen peroxide before it is applied to the wound, it should be applied to the wound shortly thereafter, e.g. not more than 30 min thereafter. This is because the reactive species generated such as peracetic acid beneficial for the treatment have a relatively short durability.

The patient to be treated with the wound dressing or other wound contact medium subjected to as described can be a human patient or an animal patient. A human patient is preferred.

The invention also relates to a kit comprising

a) a wound dressing or other wound contact medium containing an acetyl donor compound according to the invention, and
b) a means of applying hydrogen peroxide configured to apply hydrogen peroxide to the wound dressing or other wound contact medium.

In particular, the invention also relates to a kit comprising a wound dressing or other wound contact medium containing an acetyl donor compound and being free from peroxide compounds according to the invention, and a means of applying hydrogen peroxide configured to apply hydrogen peroxide to the wound dressing or other wound contact medium.
The wound dressing or other wound contact medium according to the invention, and the means of applying hydrogen peroxide have been described above so that reference is made thereto. The means of applying hydrogen peroxide is capable of generating a plasma stream that can be directed to the wound dressing or other wound contact medium to be treated. The means of applying hydrogen peroxide is capable of activating the acetyl donor compound of the wound dressing or other wound contact medium to produce peracetic acid in situ. This is effected by reaction of the hydrogen peroxide with the acetyl donor compound. The means of applying hydrogen peroxide to the wound dressing or wound contact medium is preferably selected from
a means of applying hydrogen peroxide by spraying or soaking,
a means of applying hydrogen peroxide by padding with a hydrogen peroxide containing wipe or sterile pad,
a means of applying hydrogen peroxide by drip application,
a means of applying hydrogen peroxide by striping by passing over a series of holes in an applicator bar delivering the solution,
a means of applying hydrogen peroxide by printing a solution of gel containing hydrogen peroxide by gravure or other techniques,
a means of applying hydrogen peroxide in form of a vapour or mist.

The invention is also directed to a process for preventing and/or treating a wound infection in a patient comprising applying a wound dressing or other wound contact medium containing an acetyl donor to a wound according to the invention to a wound of the patient, and subjecting the wound dressing or other wound contact medium to a means of applying hydrogen peroxide before and/or after it is applied to the wound.

In particular, the invention is also directed to a process for preventing and/or treating a wound infection in a patient comprising applying a wound dressing or other wound contact medium containing an acetyl donor compound and being free from peroxide compound, according to the invention to a wound of the patient, and subjecting the wound dressing or other wound contact medium to a means of applying hydrogen peroxide before and/or after it is applied to the wound.
In the process, a means of applying hydrogen peroxide capable of generating a plasma stream that can be directed to the wound dressing or other wound contact medium to be treated can be used. The wound dressing or other wound contact medium according to the invention, the means of applying hydrogen peroxide to the wound dressing or other wound contact medium as well as the process steps have been described above so that reference is made thereto. The means of applying hydrogen peroxide is capable of activating the acetyl donor compound of the wound dressing or other wound contact medium to produce peracetic acid in situ. This is effected by reaction of the hydrogen peroxide with the acetyl donor compound. The means of applying hydrogen peroxide is preferably selected from
a means of applying hydrogen peroxide by spraying or soaking,
a means of applying hydrogen peroxide by padding with a hydrogen peroxide containing wipe or sterile pad,
a means of applying hydrogen peroxide by drip application,
a means of applying hydrogen peroxide by striping by passing over a series of holes in an applicator bar delivering the solution,
a means of applying hydrogen peroxide by printing a solution of gel containing hydrogen peroxide by gravure or other techniques,
a means of applying hydrogen peroxide in form of a vapour or mist.

The patient can be a human or an animal. A human patient is preferred.

1. A wound dressing or other wound contact medium containing an acetyl donor compound, and being free from peroxide compounds.

2. The wound dressing or other wound contact medium according to claim 1, in which the acetyl donor compound is encapsulated within polymeric particles.

3. The wound dressing or other wound contact medium according to claim 1, in which the particles are produced by a thermally induced phase separation (TIPS) process.

4. The wound dressing or other wound contact medium according to claim 2, in which the wound dressing comprises a dressing matrix to which the particles are attached, preferably via a linking group.

5. The wound dressing or other wound contact medium according to claim 1, in which the acetyl donor compound is selected from tetraacetylethylenediamine (TAED), methyl cellulose encapsulated TAED or encapsulated donors, acetyl salicylic acid (ASA), diacetyl dioxohexahydratriazine (DADHT), tetraacetyl glycoluril, acetyl urea. di-acetyl urea, tri-acetyl urea, pentaacetyl glucose (PAG), tetraacetyl glycoluril (TAGU), acetyl phosphate, acetyl imidazole, acetyl CoA, acetic anhydride, compounds containing a hemiacetal group, acetic acid, di-, acetylmorphine, pyruvate, acetyl chloride, acetyl-caprolactam, N′N′-diacetyl-N′N′-dimethyl urea, or a combination of two or more thereof.

6. The wound dressing or other wound contact medium according to claim 1, in which the acetyl donor compound selected from tetraacetylethylenediamine (TAED), pentaacetyl glucose (PAG), acetyl salicylic acid (ASA), or a mixture of these.

7. A kit comprising
a) a wound dressing or other wound contact medium, in particular according to claim 1, containing an acetyl donor compound and
b) a means of applying hydrogen peroxide to the wound dressing capable of activating the acetyl donor compound of the wound dressing or other wound contact medium to produce peracetic acid in situ.

8. The kit according to claim 7, wherein the means of applying hydrogen peroxide to the wound dressing is selected from
a means of applying hydrogen peroxide by spraying or soaking,
a means of applying hydrogen peroxide by padding with a hydrogen peroxide containing wipe or sterile pad,
a means of applying hydrogen peroxide by drip application,
a means of applying hydrogen peroxide by striping by passing over a series of holes in an applicator bar delivering the solution,
a means of applying hydrogen peroxide by printing a solution of gel containing hydrogen peroxide by gravure or other techniques,
a means of applying hydrogen peroxide in form of a vapour or mist.

9. A wound dressing or other wound contact medium, in particular according to claim 1, containing an acetyl donor compound, for use in the prevention and/or treatment of wound infection in a patient comprising applying the wound dressing or other wound contact medium to a wound of the patient, and subjecting the wound dressing or other wound contact medium to a means of applying hydrogen peroxide to the wound dressing so that the acetyl donor compound of the wound dressing or other wound contact medium is activated by the hydrogen peroxide to produce peracetic acid in situ.

10. The wound dressing or other wound contact medium for use according to claim 9, wherein the means of applying hydrogen peroxide to the wound dressing is selected from
a means of applying hydrogen peroxide by spraying or soaking,
a means of applying hydrogen peroxide by padding with a hydrogen peroxide containing wipe or sterile pad,
a means of applying hydrogen peroxide by drip application,
a means of applying hydrogen peroxide by striping by passing over a series of holes in an applicator bar delivering the solution,
a means of applying hydrogen peroxide by printing a solution of gel containing hydrogen peroxide by gravure or other techniques,
a means of applying hydrogen peroxide in form of a vapour or mist.

11. The wound dressing or other wound contact medium for use according to claim 9, wherein the wound dressing or other wound contact medium is subjected to a means of applying hydrogen peroxide to the wound dressing before and/or after it is applied to the wound.

12. The wound dressing or other wound contact medium for use according to claim 9, wherein the patient is a human or an animal.

13. A process for preventing and/or treating a wound infection in a patient comprising applying a wound dressing or other wound contact medium, in particular according to claim 1, containing an acetyl donor compound, to a wound of the patient, and subjecting the wound dressing or other wound contact medium to a means of applying hydrogen peroxide to the wound dressing before and/or after it is applied to the wound so that the acetyl donor compound of the wound dressing or other wound contact medium is activated by the hydrogen peroxide to produce peracetic acid in situ.

14. The process according to claim 13, wherein the means of applying hydrogen peroxide to the wound dressing is selected from
a means of applying hydrogen peroxide by spraying or soaking,
a means of applying hydrogen peroxide by padding with a hydrogen peroxide containing wipe or sterile pad,
a means of applying hydrogen peroxide by drip application,
a means of applying hydrogen peroxide by striping by passing over a series of holes in an applicator bar delivering the solution,
a means of applying hydrogen peroxide by printing a solution of gel containing hydrogen peroxide by gravure or other techniques,
a means of applying hydrogen peroxide in form of a vapour or mist, sand/or wherein the patient is a human or an animal.
